(19) United States
(12) Patent Application Publication
(10) Pub. No.: US 2023/0270813 A1
(43) Pub. Date: Aug. 31, 2023

(54) TREATMENT OF CORONAVIRUS AND BACTERIAL INFECTION WITH HOUTTUYNIA CORDATA EXTRACT AND COMBINED HOUTTUYNIA CORDATA AND PHYLLANTHUS URINARIA

(71) Applicants: Jenny LEE, Sydney (AU); Helen Chui Lan CHAI, Sydney (AU)

(72) Inventors: Jenny LEE, Sydney (AU); Helen Chui Lan CHAI, Sydney (AU)

(21) Appl. No.: 18/156,011

(22) Filed: Jan. 18, 2023

(30) Foreign Application Priority Data
Jan. 18, 2022 (AU) 2022900085
Jan. 9, 2023 (AU) 2023900041

(51) Int. Cl. A61K 36/78; A61K 36/185; A61K 9/00; A61P 31/14

(52) U.S. Cl. CPC A61K 36/78; A61K 36/185; A61K 9/0014; A61P 31/14

(57) ABSTRACT

A method for treating coronavirus infection and bacterial infection in a subject in need thereof, including: applying to the subject a topical solution containing a therapeutically effective amount of Phyllanthus urinaria (PU) extract or one of its physiologically acceptable forms; and administering to the subject a composition comprising a therapeutically effective amount of Houttuynia cordata (HC) extract or one of its physiologically acceptable forms.

### FIELD

The present invention relates to a treatment of serial viruses and bacteria using compounds from plant extracts.

In particular the present invention relates to methods and compositions for use in treatment of Sar-Cov-2, Staphylococcus aureus, E. coli, urine infection, pneumonia, influenza, shigella, typhoid bacillus, Streptococcus, Moraxella catarrhalis, Eco virus, respiratory infection, bronchitis, tuberculosis, adenovirus and rhinovirus and the use of organic compounds from plant extracts in methods of therapy and the manufacture of medicaments as well as compositions containing these compounds.

The invention has been developed primarily in the use of plant extracts and herbal compounds for treating Sar-Cov-2, Staphylococcus aureus, E. coli, urine infection, pneumonia, influenza, shigella, typhoid bacillus, Streptococcus, Moraxella catarrhalis, Eco virus, respiratory infection, bronchitis, tuberculosis, adenovirus and rhinovirus, and will be described hereinafter with reference to this application. However, it will be appreciated that the invention is not limited to this particular field of use.

### BACKGROUND

COVID-19 is an infectious respiratory illness caused by the virus severe respiratory syndrome coronavirus 2 (SARS-CoV-2). The disease was first identified in December 2019 and has spread throughout the world, causing a pandemic

Common symptoms of COVID-19 are fever, cough, and shortness of breath and with a mortality rate of around 4-5%, it is more than 10 times lethal than the flu. The virus is especially threatening to public health, is highly contagious and can be spread by asymptomatic carriers or by those who are pre-symptomatic.

While anyone is susceptible to COVID-19, the ones over 60 or with pre-existing conditions, such as hypertension, obesity, asthma, or diabetes, are more vulnerable to severe symptoms. While vaccines are available, no effective antiviral therapy or prophylaxis treatment is available hence the search for effective therapies to treat SARS-CoV-2 is to date of paramount importance.

One object of the invention is to provide a medicine for treatment of SARS-CoV-2.

The present invention seeks to provide an improved treatment for SARS-CoV-2, which will overcome or substantially ameliorate at least one or more of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY

In a first aspect of the invention there is provided a method for treating coronavirus infection and bacterial infection in a subject in need thereof comprising administering to the subject a composition comprising a therapeutically effective amount of Houttuynia cordata extract or one of its physiologically acceptable salts.

In a preferred embodiment, the composition is administered in an oral dosage form in an amount of 10 ml to 25 ml three times daily.

The composition can comprise between about 100 mg to 500 mg of Houttuynia cordata liquid extract.

The extract containing organic compound, activates a biochemical reaction for cell extension and strengthen metabolism, particularly impacted to work without causing an additional harm to the benign cells, intestinal organs and healthy immune cells and to eradicate this epidemic disease.

The composition can contain additional therapeutic agents including for example, vitamin C, vitamin D, melatonin, anti-inflammatory therapy, antivirals and combinations thereof.

The composition can be administered orally, topically, intranasally, systemically, intravenously, intramuscularly, subcutaneously and the like.

The pharmaceutical composition can contain regular excipients that are acceptable in the process of drug manufacturing, such as binders, fillers, preservatives, flow control agents, softeners, wetting agents, dispersing agents, emulsifiers, solvent, antioxidants and/or propellants, drug carriers.

The pharmaceutical compositions can contain one or more of the following substances as fillers: sugars and theft derivatives (lactose, modified lactose, sucrose, glucose, mannitol, modified mannitol, fructose), polysaccharides (cellulose and its derivatives, starch, modified starch), dextrin, dextrose, dextrate, maltodextrin, calcium and its salts, (phosphates, carbonates, chlorides), magnesium and its derivatives (oxide, carbonate, stearate), crospovidone, copovidone, cyclodextrines, alginic acid and its salts, saccharine and its salts, sodium and its salts (chloride, citrate, fumarate, carbonate), aspartame, lactic acid and its salts, succinic acid, ascorbic acid, tartaric acid, colloidal silicon dioxide, cyclamate, benzoic acid and its salts, parabens and their salts.

Bacteria resistance is increasingly becoming a problem as bacteria adapt and evolve becoming resistant to medicines such as antibiotic resistant which are used to treat bacterial infections. As a result, patients can suffer repeated relapse caused by increased contagious variant disease.

A benefit of the present treatment derived from the plant Houttuynia cordata is that the natural plant chemical extract is capable of killing off bacteria and virus thereby minimizing opportunity of mutation and drug resistance.

A further benefit of the present treatment is that the natural plant chemical extract visibly rouse up body interior immunity system to defense efficiency, strengthen immune cells to confront virus capability and use of molecular compound with less likelihood of severe side effects observed in conventional treatment therapies.

In a further related aspect, the present invention relates to the synthesis of plant extract, Houttuynia cordata. The reflux extraction process involves applying distilled water for cleaning of Houttuynia cordata, pressing and moved by a screw conveyor to resolution and resolve, grinding, extraction and ultra-filtrating of concentrate.

It can be seen that the invention provides the benefit of less severe side effects associated with conventional therapy, and decrease in development of infection resistance.

In yet a further related aspect of the present invention there is provided a method for treating coronavirus infection and bacterial infection in a subject in need thereof comprising: administering to the subject in a first step a therapeutically effective amount of PU extract or one of its physiologically acceptable forms; and in a second step administering to the subject a composition comprising a therapeutically effective amount of Houttuynia cordata (HC) extract or one of its physiologically acceptable forms; wherein the second step is repeated at regular intervals.

Without being bound by theory, treatment of patients presenting with a viral infection such as SARS-CoV-2 with PU as a first step in the two-step treatment regimen of the invention, lowers fever and body aches associated with the infection and acts as a precursor which surprisingly improves the effectiveness of HC. The use of a two-step treatment produces an unexpected increase in treatment effectiveness over PU or HC alone.

Phyllanthus urinaria (PU) extract or one of its physiologically acceptable salts is preferably administered topically in a topical solution applied to the subject over some or all the body in a first dosage phase. The transdermal delivery mode of administering PU allows direct absorption into the bloodstream bypassing the intestinal tract which may otherwise expose the active constituent to chemical degradation.

Preferably, the PU solution is warmed to about 15 degrees C. or body temperature before topical application. Warming can be achieved by suspending the PU extract over a source of boiled water. It is found that application of warm PU extract is more efficient in penetrating the primary barrier of the skin (the stratum corneum) and allows enough of the drug to reach systemic circulation to deeper underlying tissues to substantially reduce fever and muscle aches in a faster timeframe. Preferably, topical administration of PU solution is applied over substantially the entire body.

Once PU extract is absorbed subcutaneously and enters the body or topically via the surface of the skin, the physiological plant chemical readily assimilates and utilizes a carcinogen “kill ” capability to act on a described case suffering from severe respiratory syndrome such as coronavirus 2 (SARS-CoV-2).

In a preferred embodiment, the HC extract is administered in an oral dosage form in an amount of 10 ml to 25 ml three times daily.

The importance of this invention is to elevate the healing efficacy to completely perish the bacteria and viruses.

Other aspects of the invention are also disclosed.

### DETAILED DESCRIPTION OF THE EMBODIMENTS

Notwithstanding any other forms which may fall within the scope of the present invention, a preferred embodiment/preferred embodiments of the invention will now be described, by way of example only.

By way of example, the formulations and treatments which follow are not intended to be limiting, and are provided for illustrating the efficacy of the inventive concept on improving apoptosis of infected cells and decreasing virus resistance and diminishing side effects in vitro and in-vivo.

### Definitions

The terms “treating” and “treatment” as used herein refer to reduction in severity and/or frequency of symptoms, elimination of symptoms and/or underlying cause, prevention of the occurrence of symptoms (prophylaxis) and/or their underlying cause, and improvement or remediation of damage.

Thus, for example, the present method of “treating” a condition encompasses both prevention of the condition in a predisposed individual, treatment of the condition in a clinically symptomatic individual and treatment of a healthy individual for beneficial effect.

“Treating” as used herein covers any treatment of, repair or prevention of a condition in a mammal, particularly a human, and includes inhibiting the condition, i.e., arresting its development; or relieving or ameliorating the effects of the condition, i.e., cause regression of the effects of the condition and producing a beneficial effect.

“Prophylaxis” or “prophylactic” or “preventative” therapy as used herein includes preventing the condition from occurring or ameliorating the subsequent progression of the condition in a subject that may be predisposed to the condition, but has not yet been diagnosed as having it.

As used herein, “condition” refers to any deviation from normal health and includes a disease, disorder, defect or injury, such as injury caused by trauma, and deterioration due to inflammatory response. Conditions in which the oral composition administration is beneficial generally fall into the categories of those in which involve treatment of virus and bacteria.

### Administration

The dosage forms are administered in a substantially pure form or together with a pharmaceutically acceptable carrier for oral administration.

The composition can be administered by any suitable route, and the person skilled in the art will readily be able to determine the most suitable route and dose for the condition to be treated and the subject. The composition can be administered orally, in dosage unit formulations containing conventional non-toxic pharmaceutically acceptable carriers, adjuvants, and vehicles.

A typical daily dosage might range from about 15 ml to up to 30 ml or more.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The invention will now be described by way of reference only to the following non-limiting examples.

### EXAMPLE 1

### Determine the Effect of Treatment Regimen Using Natural Plant Chemical Extract of Houttuynia cordata on Infected Cell Activity

### Methods and Treatment

This example examines if viral loads are decimated by the natural plant chemical extract of Houttuynia cordata according to the invention in the presence of inflammatory stimuli. According to methodology of Professor Mark Willcox and post doctoral research fellow Dr Muhammad Yasir (School of Optometry & Vision Science, UNSW), affected cells were exposed to a dosage form of a plant concentrate or one of its physiologically acceptable salts obtained from Houttuynia cordata. 50 μl of Houttuynia cordata extract was added to A9 cell lines and incubated for 30 minutes. For antiviral activity; 100 μl of dilutions of virus were added to wells which has been inoculated with the compound. The virus was allowed to interact for 1 hour followed by washing and addition of agar medium. The mouse cells were then incubated for a further 3 days at 37° C. so that the virus could replicate and kill the cells. The number of plague forming units then counted. Controls wells were without addition of compound.

Results:

The compound was not cytotoxic to A9 and produced a reduction in viruses of >4.5 log 10 (>99.99%). This screening concluded that, the compound in Houttuynia cordata extract has a very promising potential to decimate the activities of the coronavirus.

### Preparation/Extraction Methods

The preparation of the specific steps Houttuynia cordata native extract of the present invention are as follows:

Step 1

Fresh aerial parts of the plant Houttuynia cordata are cleaned with distilled water; plant extract is eluted by hydrolysis; grinding and macerating; and repeated reflux extraction where liquid extract system is condensed and returned to the system until extraction is condensed and a precipitate is obtained.

### Liquid Extract—Response Relationship

Once Houttuynia cordata extract enters the body, the plant chemical extract readily assimilates and utilizes its carcinogen “kill” capability to act on the described case.

### Case Studies and Examples

Case 1

In the first example, an 89-years old man developed symptoms of cough, shortness of breath, excruciating headaches and bouts of feverish/chills conditions especially when night falls. This individual had been administered with two full doses of Covid-19 vaccines. A casual visitor in contact with this man was also developing the same symptoms and he was tested positive to Covid-19 since the visit. Both were in isolation. However, the 89-years old man, decided to try this plant-based medicine treatment 10 ml of the Houttuynia cordata extract over separate intervals in the course of a day—morning, noon and night Within half a day, this man was found doing his heavy gardening activities—when asked he replied he was feeling 100%, all the symptoms had disappeared that he had forgotten they eventuate. A vital piece of evidence was noted after administering this extract, he expelled three loads of phlegm. His Covid-19 retest came back negative. This finding suggests there is a strong potential significance in this extract for Sar-Cov-2 virus, that caused Covid-19.

Case 2

This is a 42 year old woman living with lifetime heart conditions, in medication with her prescribed heart medicine. She was also under medication for hypertension. This woman was experiencing unexplained symptoms—shortness of breath, tiredness, constant headaches, mild cough and sporadic chills. Having thought it may be the onset of a flu, the patient bought over-the-counter medicine but that did not help, situation gotten worse—difficulty breathing comfortably. She then went got tested and was diagnosed positive to Covid-19 and confined to self-isolation. With all the uncertain information provided of the vaccine safety, and fear, droved this woman to consult a Phyto-based treatment due to her confidence in plants over conventional therapy. She started administering routinely, 10 ml of Houttuynia cordata extract in the morning, 5 ml of H. cordata at noon and 20 ml H. cordata in the evening. This specific extract was administered in space apart with her existing prescribed medicines. Much to her surprise, she started to feel relieved just over a day but continue taking the extract over the 14 days isolation period. Following this isolation period, a further retest for Covid-19 gives a negative result. This observation indicates the extract, under applied regimen have produced a surprising effect without any interaction with other medicines, encoding its botanical efficacy and increases resistance in overall health.

Case 3

A man (the patient) aged in his 30's worked as an orderly at a city hospital, was diagnosed with positive Covid-19 and had to isolate for 14 days. He had received both doses of Covid-19 vaccines. He was showing symptoms of fever, phlegm-like cough, loss of smell & lethargic. His parents having an open believer in botanical medicine subsequently approached the applicant for treatment according to the invention. The patient was hesitant to accept the extract treatment initially, but fearing that extended symptoms may compromise his overall health, he began administering routinely—20 ml H. cordata in the morning, 20 ml H. cordata at noon and 25 ml H. cordata in the evening. Patient described feeling better, signs of recovery with fever diminished being the first observation, within two days. Further Covid-19 test, shows negative result. The man chose to continue isolate and routinely administered the extract for further one week after the isolation period. This finding proved there is a strong efficacy, a connection of its effectiveness against the Sar-Cov-2 virus, particularly the apoptosis activities by applying the native extract treatment under the specific regimen in the invention.

### Benefits

The chemical extract obtained from H. cordata is found effective for increased healing efficiency without adverse side effects often associated with conventional treatments.

The natural plant chemical extract from H. cordata is capable of killing bacteria and virus thereby minimizing opportunity of mutation and drug resistance.

### Preparation/Extraction Methods of PU

A method of preparing Phyllanthus urinaria (PU) of pharmaceutically concentrated synthesis infusion comprises the following steps:
i. Fresh Phyllanthus urinaria adopts distilled water for cleanse and elution, then utilizing rotary cool evaporator for drying until all water has utter evaporated and freeze dried; and
ii. Grinding and processing via reflux extraction to obtain the infusion concentration.

The synthesis concentrated solution, adding 35% ethanol was eluted to blend and to dissolve the precipitate and later boil 100 degrees for half hour.

Further Case Studies and Examples

Case 4

A middle-aged woman in her 50's, was found by her housemate lying in her bed sweating profusely with a fever of 39.7 degrees. The patient had a rapid antigen test (RAT) which showed positive to Covid-19. The patient was treated with an alcohol extraction solution of PU applied topically to the entire body twice a day, morning and evening. Prior to administering the topical PU extract, the PU extract was warmed to about 15 degrees C. by suspending over a source of steam. The patient was also administering oral extract of H. cordata at a rate of 20 ml three times daily. The patient continued to take oral HC at regular intervals for a predetermined time thereafter.

Within 48 hours, observation noted that fever had disappeared. The patient was able to feel at ease of appetite, relieved and breathe normally. Further covid-19 retest came back negative by the end of one week.

This finding suggests a potential and promising highlight in both topical administration of PU solution and oral administration of HC substantially concurrently. More specifically it demonstrated an unexpected synergy between transdermal delivery of PU, and oral administration of HC, one being an excellent precursor treatment for the other.

Case 5

In this instance, a man presented with a positive RAT test and symptoms including fever, headaches. muscular aches and irritability feelings. The patient otherwise had no Covid vaccinations and had an excellent medical history.

The patient was treated with a warm PU dosage phase by topical application His onset of fever was also saved by the PU extract solution. As his own immunity was much stronger, the warm PU extract solution took effect in less than 24 hours to clear of his fever, along with his headaches. muscular aches and irritability feelings. He was still producing a positive rapid-antigen results (RAT) next day, but his proactive application of the solution gives him the all clear on day three.

Case 6

A 24-year-old man with a number of health issues developed symptoms of sore throat, high temperature, body aches/pains and was later tested positive to Covid-19. The patient had two approved Covid vaccines prior, which had caused an adverse reaction.

The patient was treated by topical application of warm PU extract solution, applying topically to whole body twice a day, a therapeutically amount. The patient was also taking 20 ml of HC extract orally three times daily. The patient was relieved of fever after three days of the treatment and other symptoms associated with covid reduced.

Case 7

This is a situation of a woman aged 46, presented with a recurring case of Escherichia coli (E. coli) infection. The patient experienced symptoms of severe stomach cramps, bloody diarrhea, vomiting and slightly feverish each time. Having already been living on prescribed conventional medication for heart and hypertension, she wasn't ready to accept more of this medication especially it has not shown any effective recovery from her medical consultation.

The patient was treated using topical application of a warm solution of PU in a first dosage phase, with simultaneous oral administration of HC extract at a frequency of 20 ml three times per day over the course of one week.

The patient was observed with reduced fever and fatigue and able to accomplish her work week without any thought.

This documented a surprising synergistic effect between phased dosages of topical PU and oral HC to effect reduction of fever and reduced covid symptoms. The two phased dosage of PU and HC has great and fast effectiveness for control of fever intradermally and enabling effective infection control by oral administration of HC.

Case 8

A lady in her 30's with triple covid vaccination, was exposed to Covid 19 symptomatic, asymptomatic cases at a corporate social event of around 80 people. patient developed a gradual temperature up to 41.2 degrees, upper respiratory symptoms, appetite loss, fatigue, loss of concentration.

In a first dosage phase, the whole body was exposed to topical application of a warmed PU solution twice daily in therapeutically amount of PU concentration. In a second dosage phase, the patient took oral dosages of HC. This case found the treatment breaks the patient's fever and chills.

Treatment of a virus or bacterial infection by combined topical application of PU extract to reduce fever associated with subsequent oral administration of HC at regular intervals acts as a surprising anti viral improving micro flora and helps to improve immunity by killing bacteria without harming good cells which naturally fight bacteria.

### Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the botanical medicament industries.

What is claimed is:

1. A method for treating coronavirus infection in a subject in need thereof comprising administering to the subject a composition comprising a therapeutically effective amount of Houttuynia cordata extract or one of its physiologically acceptable salts.

2. The method of claim 1, wherein the composition is administered in an oral dosage form in an amount of 10 ml to 25 ml three times daily.

3. The method of claim 1 or 2, wherein the composition comprises between about 100 mg to 500 mg of Houttuynia cordata liquid extract.

4. Use of Houttuynia cordata extract or one of its physiologically acceptable salts, in the treatment of a disease or condition in a mammal such as a human, selected from Staphylococcus aureus, E. coli, urine infection, pneumonia, influenza, shigella, typhoid bacillus, Streptococcus, Moraxella catarrhalis, Eco virus, respiratory infection, bronchitis, tuberculosis, adenovirus and rhinovirus, wherein the Houttuynia cordata extract or one of its physiologically acceptable salts is formulated in a formulation comprising in the range from 0.01% to 5% of said extract, in a physiologically acceptable vehicle.

5. Use of an extract of Houttuynia cordata for the manufacture of a medicament for the prevention and/or treatment of a covid-19 infection.

6. An extract of Houttuynia cordata for use as a medicament for the treatment of a disease or condition of a mammal selected from Staphylococcus aureus, E. coli, urine infection, pneumonia, influenza, shigella, typhoid bacillus, Streptococcus, Moraxella catarrhalis, Eco virus, respiratory infection, bronchitis, tuberculosis, adenovirus and rhinovirus, wherein the extract of Houttuynia cordata is formulated in a formulation comprising in the range from 0.010% to 5% of the extract, in physiologically acceptable vehicle.

7. A method of treatment of a mammal such as a human suffering from a disease or condition selected from Staphylococcus aureus, E. coli, urine infection, pneumonia, influenza, shigella, typhoid bacillus, Streptococcus, Moraxella catarrhalis, Eco virus, respiratory infection, bronchitis, tuberculosis, adenovirus and rhinovirus, comprising administering a therapeutically effective amount of Houttuynia cordata extract or one of its physiologically acceptable salts, wherein the Houttuynia cordata extract is formulated in a formulation comprising in the range from 0.01% to 5% of the extract, in a physiologically acceptable vehicle.

8. A method for treating coronavirus infection and bacterial infection in a subject in need thereof comprising:
applying to the subject a topical solution containing a therapeutically effective amount of Phyllanthus urinaria extract or one of its physiologically acceptable forms; and
administering to the subject a composition comprising a therapeutically effective amount of Houttuynia cordata extract or one of its physiologically acceptable forms.

9. The method of claim 8 wherein the second step is repeated at regular intervals.

10. The method of claim 8 wherein Phyllanthus urinaria extract or one of its physiologically acceptable salts is administered topically in a topical solution applied to the subject over some or all the body in a first dosage phase.

11. The method of claim 8 wherein Phyllanthus urinaria extract or one of its physiologically acceptable salts is warmed to about 15 degrees C. or body temperature before topical application.

12. The method of claim 8 wherein the Houttuynia cordata extract is administered in an oral dosage form in an amount of 10 ml to 25 ml three times daily.
